(19) United States
(12) Patent Application Publication
(10) Pub. No.: US 2010/0029575 A1
(43) Pub. Date: Feb. 4, 2010

(54) N-Oxides of Kappa Receptor Peptides

(76) Inventors: Jean-Louis Junien, Sevres (FR); Pierre J.M. Riviere, San Diego, CA (US); Claudio D. Schteingart, San Diego, CA (US); Javier Sueiras Diaz, La Jolla, CA (US); Jerzy A. Trojnar, San Diego, CA (US); Todd W. Vanderah, Tucson, AZ (US); Michael E. Lewis, West Chester, PA (US)

Correspondence Address: CARA THERAPEUTICS, INC.;Intellectual Property Department, One Parrott Drive, Shelton, CT, 06484-4733, US

(21) Appl. No.: 12/300,610

(22) PCT Filed: May 25, 2007
(86) PCT No.: PCT/US07/12463

§ 371 Date: May 6, 2009

Related U.S. Application Data
(60) Provisional application No. 60/808,656, filed on May 26, 2006.

(51) Int. Cl. A61K 38/07; C07K 5/00; A61P 25/00

(52) U.S. Cl. 514/18; 530/330

(57) ABSTRACT

Certain peptides which exhibit high selectivity for the kappa opioid receptor (KOR) versus the mu opioid receptor and little or no CYP3A4 inhibitory activity including tetrapeptides of four D-isomer amino acid residues having a C-terminus which is an N-oxide-substituted amide such, as H-D-Phe-D-Phe-D-Nle-D-Arg-NH-4-picolyl-N-oxide. A preferred compound, which has an affinity for the KOR at least 1,000 times its affinity for the mu opioid receptor and an IC50 for CYP3 A4 of greater than about 10 micromolar, is H-D-Phe-D-Phe-D-Nle-D-Arg-NH-4-picolyl-N-oxide

### CROSS-REFERENCE TO RELATED APPLICATIONS

This Application claims priority to and incorporates by reference herein U.S. Provisional Application Ser. No. 60/808,656 filed May 26, 2006 and entitled “N-OXIDES OF KAPPA OPIOID RECEPTOR PEPTIDES.”

### FIELD OF THE INVENTION

The invention relates to metabolites of certain synthetic peptide amides and N-oxides of certain synthetic peptide amides, including such compounds which are highly selective kappa receptor agonists and which exhibit little or no cytochrome p450 inhibitory activity such as CYP3A4 inhibitory activity.

### BACKGROUND OF THE INVENTION

Kappa opioid receptors (KORs) are present in the brain, spinal cord, and on the central and peripheral terminals and cell bodies of the primary sensory afferents (somatic and visceral), as well as on immune cells.

KORs which are located in the brain have been shown to mediate the central analgesic effects of molecules, commonly referred to as kappa agonists, which activate such KORs. This finding led to numerous attempts (i.e. Spiradoline from Upjohn and Enadoline from Parke-Davis) to develop brain-penetrating, non-peptidic kappa agonists for use as original analgesics which would be devoid of the unwanted side effects (constipation, respiratory depression, dependence and addiction) of morphinic analogs that act on mu opioid receptors (MORs). The analgesic activity, as well as the lack of mu-opioid side effects, of this class of compounds has been established both in animals and humans. However, some systemic kappa agonists were also shown to induce specific side effects such as diuresis, sedation and dysphoria, mediated through kappa receptors located in the brain, which resulted in the discontinuation of their development.

In addition to such centrally mediated analgesia, stimulation of KORs located either in the periphery or in the spinal cord may also produce analgesia. However, neither peripheral nor spinal KORs were associated with any of the side effects of systemic kappa agonists. Therefore, as long as it is possible to create kappa receptor opioid agonists that do not enter the brain (following either peripheral or spinal administration), it should be possible to obtain safe and original analgesics.

It is now considered that peptidic opioid agonists that are selective for the KOR should be ideal for this purpose because they are likely, at the most, to only poorly enter the brain after either peripheral or spinal administration; therefore, they are expected to be devoid of central side effects. U.S. Pat. No. 5,610,271 discloses tetrapeptides containing four D-isomer amino acid residues that bind to KORs, and U.S. Pat. No. 5,965,701 discloses certain synthetic peptides that have a long duration of peripheral activity. Parenteral (i.v., i.m., s.c. epidural, topical or local) routes of administration may be suitable for this class of compounds to treat pain in conditions associated with inflammation, such as rheumatoid arthritis, or post-operative pain, such as that resulting from eye surgery, dental surgery, articulation surgery, abdominal surgery, childbirth and cesarean section. Furthermore, alleviation of abdominal postsurgery symptoms (digestive ileus) is presently considered to be a major therapeutic target of peptidic kappa agonists. These symptoms include motility disorders such as bloating, nausea, and intestinal transit inhibitions associated with sensitivity disorders, such as pain possibly induced by distension. Such motor disturbances are considered to be the consequence of a prior alteration of visceral sensitivity resulting from nerve sensitization by local inflammatory process, and it has been shown in animal models that compounds which block pain may also reverse motor impairments (Riviere et al., Gastroenterology, 104:724-731, 1993). Indeed, non-peptidic kappa agonists were shown to produce antinociception in experimental ileus that was associated with a restoration of normal motor functions. Such provides a rationale for developing non-brain-penetrating kappa agonists for treatment of post-operative pain and digestive ileus (Friese et al., Life Sciences, 60(9):625-634, 1997). Because such kappa agonists generally do not exhibit a constipating or antitransit side effect, they have a major advantage for this indication compared to morphine-like compounds.

It has also been shown that kappa agonists produce peripheral antinociception in models of intestinal as well as colonic hyperalgesia induced by mild and local inflammation (Diop et al., Eur. J. Pharm., 271:65-71, 1994). As a result, Irritable Bowel Syndrome (IBS), which includes exaggerated visceral pain due to a visceral hypersensitivity possibly linked to a local inflammation, is also a target for a peripheral kappa agonist (Junien and Riviere, Alimentary Pharmacology and Therapeutics, 9:117-126, 1995).

In addition to the gastrointestinal tract, other viscera showing a pathological condition that involves activation and/or sensitization (i.e. local inflammation) of primary sensory afferents are also considered to represent appropriate targets for such a kappa receptor opioid. Examples of these conditions where kappa receptor opioids can be used include urinary incontinence due to bladder inflammation (cystitis), dysmenorrhea, vasomotor rhinitis, ocular inflammation, and kidney or bladder stone-induced pain.

It was established in somatic tissues that kappa agonists also block neurogenic inflammation by inhibiting the release of substance P from primary sensory afferents. Assuming such activity is also present in GI and visceral tissues, peripheral kappa agonists would be expected to have an ameliorating effect in conditions where pain or visceral hypersensitivity is associated with neurogenic inflammation (e.g. bladder cystitis).

Kappa opioid agonists are also known to act on the immune system and have primarily an inhibitory role on immune cells. Their effects include (i) suppression of T cell-dependent antibody production, (ii) alteration of mitogen- and antigen-induced lymphocyte proliferation, (iii) modulation of natural killer (NK) cell- and T cell-mediated cytotoxicity; (iv) chemotaxis of peripheral blood derived mononuclear cells (PBMC), and (v) alteration of PBMC function. These effects might be of interest in some specific indications, where it is important to lower the immune response.

Kappa opioid agonists are also known to ameliorate pruritis (i.e., itch) associated with a variety of conditions, e.g., uremic pruritis in patients undergoing hemodialysis, and it is expected that peripherally acting kappa agonists will be useful in treating such conditions.

In addition, kappa opioid agonists are known to produce a free water diuresis, or aquaresis. Without wishing to be bound by theory, this electrolyte-sparing diuresis is believed to be due to an effect on kappa opioid receptors in the kidney or in the posterior pituitary and/or basomedial hypothalamic neurons lying outside the blood-brain barrier, which can result in an inhibition of the release and/or biological effects of vasopressin. It is expected, therefore, that peripherally acting kappa agonists will be useful in the treatment of euvolemic hyponatremia, a potentially life-threatening condition that occurs when the body's blood sodium level falls below normal. Euvolemic hyponatremia, which occurs when total body water increases with little increase in sodium, is most often associated with conditions such as the syndrome of inappropriate antidiuretic hormone (SIADH), adrenal insufficiency, pulmonary disorders, hypothyroidism, certain cancers and the use of certain drugs (such as some antidepressants). Hyponatremia often results from elevated levels of the hormone arginine vasopressin (AVP), which regulates water and salt balance in the body. It is the most common electrolyte disorder in hospitalized patients and one of the most difficult to treat. Current therapeutic approaches have focused on arginine vasopressin (AVP) receptor antagonists, such as conivaptan. However, because conivaptan has CYP3A4 inhibitory activity, it cannot be co-administered with potent CYP3A4 inhibitors, such as ketoconazole, itraconazole, clarithromycin, ritonavir, and indinavir, and there is a need for therapeutic agents that do not have this limitation. Peripherally acting kappa agonists of the invention are expected to have clinical utility to improve the imbalance of salt and water in disorders such as congestive heart failure, liver cirrhosis and conditions causing SIADH.

Particularly desired are peptides which exhibit high affinity for the KOR versus the MOR, but with little or no cytochrome inhibitory activity on isoenzymes/isoforms of cytochrome P450 monooxygenases (CYP450), particularly the CYP3A4 isozyme/isoform. These characteristics are desirable because many important drug interactions involve these isoenzymes, which are located in the intestines, liver, lung, kidney and brain. Drug metabolism occurs in two phases: Phase I involves oxidation, reduction, and hydrolysis, while Phase II involves synthesis and conjugation. The CYP 450 isoenzymes are involved in Phase I oxidative reactions. CYP 450 drug interactions generally result from one of two processes: inhibition and induction. Induction means that a substance stimulates the synthesis of the enzyme and metabolic capacity is increased. Inhibition means competitive binding at an enzyme's binding site(s). A drug with a high affinity for a CYP 450 isoenzyme will slow the metabolism of any low affinity drug that would normally be metabolized by that isoenzyme, potentially resulting in accumulation of the drug in the body to toxic levels. CYP 3A4 is the most abundant CYP 450 isoenzyme in humans and is responsible for the metabolism of the widest range of drugs, including amiodarone, carbamazepine, amitriptyline, nefazadone, sertraline, various benzodiazapines, Ca channel blockers, astemizole, terfenidine, buspirone, ciprofloxacin, lansoprazole, ‘statins’, and cisapride. Thus, these drugs must be administered with great care if patients are also receiving drugs that act as potent inhibitors of CYP3A4, such as macrolide antibiotics, fluoxetine, fluvoxamine, grapefruit juice, indinavir, itraconazole, ketoconazole, and ritonavir.

Other important CYP 450 isoenzymes include:
CYP 2D6—fluoxetine, paroxetine and quinidine are potent inhibitors of this enzyme. Drugs metabolized by CYP 2D6 include amiodarone, haloperidol, and selegiline.
CYP 1A2—erythromycin and fluvoxamine are potent inhibitors of this enzyme. Drugs metabolized by CYP 1A2 include acetaminophen, amitriptyline and propranolol.
CYP 2C9/10—SSRI antidepressants, cimetidine, zafirlukast, ‘statins’, amiodarone, fluconazole are potent inhibitors of this enzyme. Warfarin, nonsteroidal anti-inflammatory drugs, phenytoin, and angiotensin II receptor antagonists are metabolized by CYP 2C9/10.

Thus, it is an object of this invention to provide kappa opioid peptides that have little or no inhibitory activity at these CYP 450 enzymes, in order to increase the therapeutic utility of such peptides by avoiding potentially dangerous interactions with other drugs being taken by subjects in need of antinociception or other therapeutic benefit thereof.

### SUMMARY OF THE INVENTION

In one embodiment the invention provides in general a genus of synthetic peptide amides which exhibit high selectivity for the KOR and which do not exhibit any significant inhibition of CYP 450 enzymes. In some embodiments of the invention the lack of significant inhibition of CYP 450 enzymes is manifested by exhibiting little or no inhibitory activity of the liver metabolizing enzyme CYP3A4.

In some embodiments, the invention provides compounds comprising metabolites of synthetic peptide amides wherein (i) the metabolites can be formed from administration of the peptide amide to a mammal, (ii) the compounds have an affinity for the kappa opioid receptor which is at least 1,000 times its affinity for the mu opioid receptor, (iii) the compounds exhibit little or no cytochrome P450 enzyme inhibitory activity, and (iv) the peptide amide has the formula:

H-Xaa₁-Xaa₂-Xaa₃-Xaa₄-Q

wherein Xaa₁ is selected from the group consisting of D-Phe wherein the phenyl group is optionally substituted with NO₂, F, Cl or CH₃, D-Phe wherein the amino acid alpha carbon is methyl substituted, D-Tyr, D-Tic, D-Acp, D-2-Thi, or D-3-Thi; Xaa₂ is selected from the group consisting of D-Phe wherein the phenyl group is optionally substituted with NO₂, F, Cl, 3,4-dichloro or CH₃, D-1Nal, D-2Nal, D-Tyr or D-Trp; Xaa₃ is selected from the group consisting of D-Nle, D-Leu, D-Leu wherein the amino acid alpha carbon is methyl substituted, D-Hle, D-Met, D-Val, D-Phe or D-Acp; Xaa₄ is selected from the group consisting of D-Arg, D-Har, D-nArg, D-Lys, D-Lys(Ipr), D-Arg(Et₂), D-Har(Et₂), D-Amf(G), D-Dbu, D-Orn, D-Orn wherein the amino acid alpha carbon is methyl substituted, or D-Orn(Ipr), with G being H or amidino; and Q is NR₁R₂, piperidinyl, 4-hydroxy piperidinyl, 4-oxo piperidinyl, piperazinyl, 4-mono- or 4,4-di-substituted piperazinyl or delta-ornithinyl, with R₁ being substituted benzyl, 2-thiazolyl, 2-picolyl, 3-picolyl or 4-picolyl, R₂ being H or lower alkyl.

In some embodiments of the invention the metabolites are N-oxides of the synthetic peptide amide.

In some embodiments, the invention provides compounds which are N-oxides of synthetic peptide amides having the formula:

H-Xaa₁-Xaa₂-Xaa₃-Xaa₄-Q-N-oxide

wherein Xaa₁ is selected from the group consisting of D-Phe wherein the phenyl group is optionally substituted with NO₂, F, Cl or CH₃, D-Phe wherein the amino acid alpha carbon is methyl substituted, D-Tyr, D-Tic, D-Acp, D-2-Thi, or D-3-Thi; Xaa₂ is selected from the group consisting of D-Phe wherein the phenyl group is optionally substituted with NO₂, F, Cl, 3,4-dichloro or CH₃, D-1Nal, D-2Nal, D-Tyr or D-Trp; Xaa₃ is selected from the group consisting of D-Nle, D-Leu, D-Leu wherein the amino acid alpha carbon is methyl substituted, D-Hle, D-Met, D-Val, D-Phe or D-Acp; Xaa₄ is selected from the group consisting of D-Arg, D-Har, D-nArg, D-Lys, D-Lys(Ipr), D-Arg(Et₂), D-Har(Et₂), D-Amf(G), D-Dbu, D-Orn, D-Orn wherein the amino acid alpha carbon is methyl substituted, or D-Orn(Ipr), with G being H or amidino; and Q is NR₁R₂, piperidinyl, 4-hydroxy piperidinyl, 4-oxo piperidinyl, piperazinyl, 4-mono- or 4,4-di-substituted piperazinyl or delta-ornithinyl, with R₁ being substituted benzyl, 2-thiazolyl, 2-picolyl, 3-picolyl or 4-picolyl, R₂ being H or lower alkyl.

Some embodiments feature compounds which are amide-N-oxides such as the N-oxides of 2-picolylamide, 3-picolylamide, 4-picolylamide, and piperazineamide.

### BRIEF DESCRIPTION OF THE FIGURES

FIG. 1 is a graph showing the timeline for the steps performed according to the Mouse Acetic Acid Writhing Test Method.

FIG. 2 is a graph showing Antinociceptive Effect of intravenous injection of Peptide I on Visceral Pain using the Mouse Acetic Acid Writhing Test Method. Percent antinociception as measured using the Mouse Acetic Acid Writhing Test Method is plotted against dose on a log scale with a linear fit based on data points at dosages of 10, 30, and 100 micrograms peptide 1/kilogram of body weight.

### DETAILED DESCRIPTION OF THE INVENTION

The nomenclature used to define the peptides is that specified by Schroder & Lubke, The Peptides, Academic Press, 1965, wherein, in accordance with conventional representation, the N-terminus appears to the left and the C-terminus to the right. Where an amino acid residue has isomeric forms, it is the L-isomer form of the amino acid that is being represented herein unless otherwise expressly indicated.

In some embodiments, the invention provides peptides which are selective for the KOR and not only exhibit a strong affinity for the KOR but exhibit little or no CYP3A4 inhibitory activity or exhibit little or no inhibitory activity of another CYP 450 enzyme. In certain embodiments, kappa selective opioid peptides of the invention have at least 1,000 times greater binding affinity for the KOR than the MOR, with many compounds having at least 10,000 times greater affinity, and with some compounds exhibiting an affinity of 20,000 or more times greater. However, for many indications it is important that, along with such high selectivity, the kappa agonists should exhibit both a lack of cytochrome P450 inhibitory activity as well as in vivo antinociceptive activity.

As generally indicated hereinbefore, the invention provides in certain embodiments a genus of D-isomer tetrapeptides having the formula which follows:

H-Xaa₁-Xaa₂-Xaa₃-Xaa₄-Q-N-oxide

wherein Xaa₁ is (A)D-Phe, (C^{alpha} Me)D-Phe, D-Tyr, D-Tic, D-Acp, D-2-Thi, or D-3-Thi, with A being H, NO₂, F, Cl or CH₃; Xaa₂ is (A′)D-Phe, D-1Nal, D-2Nal, D-Tyr or D-Trp, with A′ being A or 3,4Cl₂; Xaa₃ is D-Nle, (B)D-Leu, D-Hle, D-Met, D-Val, D-Phe or D-Acp with B being H or C^{alpha} Me; Xaa₄ is D-Arg, D-Har, D-nArg, D-Lys, D-Lys(Ipr), D-Arg(Et₂), D-Har(Et₂); D-Amf(G), D-Dbu, (B)D-Orn or D-Orn(Ipr), with G being H or amidino; and Q is NR₁R₂, (C)piperidinyl, piperazinyl, mono- or di-substituted piperazinyl or delta-ornithinyl, with R₁ being 2-thiazolyl, 2-picolyl, 3-picolyl or 4-picolyl, R₂ being H or lower alkyl; and C being H, 4-hydroxy or 4-oxo.

By D-Nle is meant D-norleucine, and D-Hle represents D-homoleucine. D-Har represents D-homoarginine, and D-nArg represents D-norarginine which is one carbon shorter than D-Arg. By D-Nal is meant the D-isomer of alanine which is substituted by naphthyl on the beta-carbon. Preferably, D-2Nal is employed, i.e. the attachment to naphthalene is at the 2-position on the ring structure; however, D-1Nal may also be used. The abbreviations D-Cpa and D-Fpa are used to represent, respectively, chloro-D-Phe and fluoro-D-Phe, with D-4Cpa, D-2Fpa, D-3Fpa and D-4Fpa being preferred. D-Npa means nitro-D-Phe, and D-Mpa is used to represent methyl D-Phe. D-3,4Cpa means 3,4-dichloro-D-Phe. D-Acp represents D-Ala(cyclopentyl). D-Orn represents D-ornithine, and D-Dbu represents alpha, gamma-diamino butyric acid. CML represents C^{alpha} methyl Leu, and CMP and CMO represent C^{alpha} Me Phe and C^{alpha} Me Orn. By D-4Amf is meant D-4(NH₂CH₂)Phe, and by D-Gmf is meant Amf(amidino) which represents D-Phe where the 4-position is substituted with CH₂NHC(NH)NH₂. Amd represents amidino, and the symbol D-Amf(Amd) is also used. By D-Tic is meant D-1,2,3,4-tetrahydroisoquinoline-3-carboxylic acid. In Ala(Thi), Thi represents the thienyl group, which is preferably linked at its 2-position to alanine, although 3-thienyl is an equivalent. By Ily and Ior are respectively meant isopropyl Lys and isopropyl Orn where the side chain amino group is alkylated with isopropyl.

By lower alkyl is meant C₁ to C₆, and preferably C₁-C₄ but including cyclopropyl and cyclobutyl. Me, Et, Pr, Ipr, Bu, Pn and Bzl are used to represent methyl, ethyl, propyl, isopropyl, butyl, pentyl and benzyl. By Cyp is meant cyclopropyl, and by Cyb is meant cyclobutyl. Although the linkage is preferably to one end of an alkyl chain, the linkage may be elsewhere in the chain, e.g. 3-pentyl which may also be referred to as ethylpropyl. 4Nbz and 4Abz represent 4-nitrobenzyl and 4-aminobenzyl. By 2-, 3- and 4-picolyl (Pic) are meant methylpyridine groups with the attachment being via a methylene in the 2-, 3- or 4-position. 4Ahx is used to represent 4-aminocyclohexyl, and hEt is used to represent hydroxyethyl, i.e.—CH₂CH₂OH. Aeb is used to represent 4-(2-amino-2-carboxyethyl)benzyl, as shown in U.S. Pat. No. 5,965,701. By Pip is meant piperidinyl, and by 4-HyP and OxP are meant 4-hydroxypiperidinyl and 4-oxo-piperidinyl. By Ppz is meant piperazinyl. Ecp represents 4-ethylcarbamoylpiperazinyl; quaternary ammonium moieties, such as 4-dimethyl piperazinyl (Dmp) or other di-lower alkyl substitutions, may also be used. Substituted benzyl is preferably 4-aminobenzyl, and by 2-Tzl is meant 2-thiazolyl, as shown in U.S. Pat. No. 5,965,701. By Dor is meant delta-ornithinyl where the side chain amino group of L-ornithine is connected by an amide bond to the-C-terminus. Abbreviations for chemical moieties used herein are additionally described in the following chart:

| Abbreviation | Definition |
|---|---|
| D-Phe | D-phenylalanine |
| D-Tyr | D-tyrosine |
| D-Tic | D-1,2,3,4-tetrahydroisoquinoline-3carboxylic acid |
| D-Ala | D-alanine |
| D-1Na1 | D-Alanine substituted by naphthyl on the beta carbon with |
| | the point of attachment at the 1-position on the naphthyl |
| | ring structure |
| D-2Na1 | D-Alanine substituted by naphthyl on the beta carbon with |
| | the point of attachment at the 2-position on the naphthyl |
| | ring structure |
| D-Trp | D-tryptophan |
| D-Nle | D-norleucine |
| D-Leu | D-leucine |
| D-Hle | D-homoleucine |
| D-Met | D-methionine |
| D-Val | D-valine |
| D-Arg | D-arginine |
| D-Har | D-homoarginine |
| D-nArg | D-norarginine |
| D-Lys | D-lysine |
| D-Ily | Isopropyl-D-lysine |
| D-Arg(Et₂) | diethyl-D-arginine |
| D-Har(Et₂) | diethyl-D-homoarginine |
| D-Amf | D-(NH₂CH₂)-Phenylalanine |
| D-Gmf | D-(CH₂NHC(NH)NH₂)-Phenylalanine |
| D-Dbu | Alpha, gamma-diamino butyric acid |
| D-Orn | D-ornithine |
| D-Ior | Isopropyl-D-ornithine |
| Aeb | 4-(2-amino-2-carboxyethyl)benzyl |
| Ppz | piperazinyl |
| Pcp | 4-phenyl carbamoyl piperazin-1-yl |
| Aao | 8-(acetylamino)-3,6-dioxaoct-1-yl |
| Aoo | 8-amino-3,6-dioxaoct-1-yl |
| Hoh | 6-(L-hydroorotylamino)-hex-1-yl; L-hydroorotic acid is |
| | C₄N₂H₅(O)₂-COOH |
| Ghx | 6-(D-gluconylamino)-hexyl |
| Gao | 6-(D-gluconylamino)-3,6-dioxaoct-1-yl |
| D-4Fpa | 4-fluoro-D-phenylalanine |
| D-4Cpa | 4-chloro-D-phenylalanine |
| D-3, 4Cpa | 3,4-dichloro-D-phenylalanine |
| D-CML | C^{α}-methyl-D-Leucine |
| D-Acp | D-Ala(cyclopentyl) |
| Mor | Morpholinyl |
| Tmo | thiomorpholinyl |
| Pip | Piperidinyl |
| 4-HyP | 4-hydroxy piperidin-1-yl |
| OxP | 4-oxo-piperidin-1-yl |
| Me | Methyl |
| Et | Ethyl |
| Pr | Propyl |
| Bu | Butyl |
| HEt | Hydroxyethyl (i.e., —CH₂CH₂OH) |
| Cyp | Cyclopropyl |
| Bzl | Benzyl |
| D-2Fpa | 2-fluoro-D-phenylalanine |
| D-2-Thi | 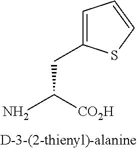 |
| D-3-Thi | 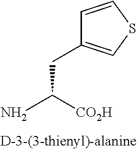 |
| 4Pic | 4-picolyl |
| C^{α}methyl | Methyl attached to the alpha carbon of an amino acid |

In some embodiments, D-Phe or substituted D-Phe is in the 1-position. The phenyl ring may be substituted at the 2-, 3- and/or 4 ring-positions, and commonly substitutions by chlorine or fluorine at the 2 or 4 ring-position are preferred. In some embodiments, the alpha-carbon atom may also be methylated. Other equivalent residues which resemble D-Phe may also be used, and these include D-Acp, D-Ala(thienyl), D-Tyr and D-Tic. The 2-position residue is also preferably D-Phe or substituted D-Phe with such substitutions preferably including a substituent on the 4-position carbon of the phenyl ring or the 3- and 4 ring-positions. Alternatively, D-alanine substituted by naphthyl can be used, as well as D-Trp and D-Tyr. The 3-position residue is preferably occupied by an amino acid residue such as D-Nle, D-Leu, D-CML, D-Hle, D-Met or D-Val; however, D-Acp or D-Phe may also be used. D-Arg and D-Har; which may be substituted with diethyl, are generally preferred for the 4-position residue; however, D-nArg and other equivalent residues may be used, such as D-Lys or D-Orn (either of which can have its omega-amino group alkylated as by isopropyl or have its alpha-carbon group methylated). Moreover, D-Dbu, D-4Amf (which is preferably substituted with amidino), and D-His may also be used.

In some embodiments, CYP3A4 inhibitory activity of tetrapeptide compounds having a substituted amide at the C-terminus, such as those disclosed in U.S. Pat. No. 5,965,701, is surprisingly and substantially attenuated through the preparation of a derivative of such a compound with the formation of an N-oxide moiety. Such N-oxides may be in the form of picolyl N-oxides, as well as other equivalent residues, such as substituted benzyl. Generally, picolyl N-oxide substitutents are preferred for single substituted amides. Instead of a single substituted amide, a dialkyl substitution, e.g. diethylamino, is an alternative; however, preferably such a disubstituted C-terminus is occupied by a piperidinyl N-oxide or 4-piperazinyl N-oxide moieties.

Without being bound to any particular theory, KOR binding is generally believed to be an attribute of the amino acid sequence of a tetrapeptide, and consequently some embodiments provide selective kappa receptor opioid peptides should which exhibit a binding affinity to the kappa receptor such that its IC₅₀, under the assay conditions described herein, is equal to about 10 nM or less.

In some embodiments, the invention provides a subgenus of opioid peptides having the formula:

H-Xaa₁-Xaa₂-Xaa₃-Xaa₄-Q-N-oxide

wherein Xaa₁ is D-Phe (unsubstituted or substituted by C^{alpha} Me, 2F, 4F or 4Cl) D-Acp, D-2-Thi, or D-3-Thi; Xaa₂ is (A′)D-Phe, D-1Nal, D-2Nal or D-Trp, with A′ being H, 4F, 4Cl, 4NO₂ or 3,4Cl₂; Xaa₃ is D-Nle, D-Leu, D-CML, D-Met or D-Acp; Xaa₄ is D-Arg, D-Arg(Et₂), D-Lys, D-Ily, D-Har, D-Har(Et₂), D-nArg, D-Orn, D-Ior, D-Dbu, D-Amf, and D-Amf(Amd); and Q is NR₁R₂, Pip, 4-HyP, OxP or Ppz, with R₁ being Me, Et, Pr, Bu, hEt, Cyp, Bzl or 4-picolyl, and R₂ being H or Et.

Another embodiment of the invention provides a subgenus of kappa opioid peptides having the formula:

H-Xaa₁-Xaa₂-Xaa₃-Xaa₄-Q-N-oxide

wherein Xaa₁ is D-Phe, D-4Fpa, D-2Fpa, D-4Cpa, D-Acp or D-Ala(Thi); Xaa₂ is D-Phe, D-4Fpa, D-4Cpa, D1Nal, D-2Nal or D-Trp; Xaa₃ is D-Nle, D-Met, D-CML or D-Leu; Xaa₄ is D-Arg, D-Lys, D-Har, D-nArg or D-Orn; and Q is NR₁R₂, Pip, 4-HyP or Ppz, with R₁ being Et, Pr, Bu, Cyp, hEt, Bzl or 4-Pic, and R₂ being H or Et.

Another embodiment of the invention provides a subgenus of kappa opioid peptides having the formula:

H-Xaa₁-Xaa₂-Xaa₃-Xaa₄-Q-N-oxide

wherein Xaa₁ is D-Phe, D-4Fpa, D-2Fpa, D-Acp or D-Ala(2Thi); Xaa₂ is (A)D-Phe, D-1Nal, D-2Nal or D-Trp, with A being 4F or 4Cl; Xaa₃ is D-Nle, D-Met or D-Leu; Xaa₄ is D-Arg, D-Har, D-nArg, D-Lys, D-Orn or D-Amf(Amd); and Q is NHR₁, Pip or Ppz, with R₁ being Et, Pr or 4Pic.

In some embodiments, Xaa₁ and Xaa₂ are D-Phe, Xaa₃ is D-Nle or D-Leu, and Xaa₄ is D-Arg or D-Orn. Further in some embodiments, the invention provides a compound H-D-Phe-D-Phe-DNle-D-Arg-NH-4-picolyl N-oxide optionally including or excluding any pharmaceutically acceptable counterions and wherein acetate ions are an example of a pharmaceutically acceptable counterion.

In some embodiments, the opioid peptides of the invention are believed to have antinociceptive in vivo activity as well as substantially reduced inhibition of cytochrome P450 enzymes as a result of incorporating an N-oxide substituted amide at the C-terminus of the position-4 amino acid residue. This particular unexpected attribute renders such peptides particularly valuable as certain of them are not only active as analgesics, but also have essentially no CYP3A4 inhibitory activity, and therefore will not cause cytochrome CYP3A4 inhibition-based drug interactions. Certain tetrapeptides of some of the foregoing embodiments of the invention that are prepared without an N-oxide substituted C-terminal amide also demonstrate high selectivity for the KOR, as compared to the MOR and they may also exhibit CYP3A4 inhibitory activity at concentrations under 10 micromolar. It is fully expected, however, that many of such opioid peptides will exhibit even lower CYP3A4 inhibitory activity when synthesized so as to have an N-oxide substituted amide, such as 4-picolyl-N-oxide, at the C-terminus.

Although many amino acid sequences are set forth in the descriptions of the foregoing embodiments of the invention, it should be understood by those having ordinary skill in the peptide chemistry art that one or more of the recited amino acid residues might be substituted by a conservative amino acid substitution, e.g., one basic amino acid for another, or one hydrophobic amino acid for another, e.g., D-Ile for D-Leu. Likewise, various of the residues may also be modified as generally known in this art; for example, D-Phe (as earlier indicated) may be modified by incorporating a halogen or nitro group usually at the 3- or 4-position, or both, or the alpha-carbon may be methylated. Such modifications are considered to produce equivalent kappa receptor opioid peptides.

The peptides can be synthesized by any suitable method, such as by exclusively solid phase techniques or classical solution addition or alternatively by partial solid phase techniques or by fragment condensation techniques. For example, the techniques of exclusively solid-phase peptide synthesis (SPPS) are set forth in the textbook Stewart & Young, Solid-Phase Peptide Synthesis, 2nd Ed., Pierce Chemical Company, Rockford, Ill., 1984, and are exemplified by the disclosure of U.S. Pat. No. 4,105,603. The fragment condensation method of synthesis is exemplified in U.S. Pat. No. 3,972,859, and other available syntheses are exemplified by U.S. Pat. Nos. 3,842,067 and 3,862,925. Classical solution addition synthesis is described in detail in Bodanzsky et al., Peptide Synthesis, 2nd Ed., John Wiley & Sons, New York, 1976.

Common to coupling-type chemical synthesis of peptides is the protection of any labile side chain of an amino acid being coupled, and usually the protection also of the .alpha.-amino group, so that the addition takes place at the carboxyl group of the individual amino acid or dipeptide or tripeptide that is being added. Such protecting groups are well known in the art, and tert-butyloxycarbonyl (Boc), benzyloxycarbonyl(Z) and 9-fluorenylmethoxycarbonyl (Fmoc) are often used as preferred alpha-amino protecting groups in SPPS or classical solution synthesis although there are a large variety of other alpha-amino protecting groups that may alternatively be used.

When SPPS is used, the C-terminal amino-acid residue is coupled to a solid resin support such as O—CH₂-polystyrene support, O—CH₂-benzyl-polyamide resin support, —NH-benzhydrylamine (BHA) resin support, or —NH-para methylbenzhydrylamine (MBHA) resin support. The use of BHA or MBHA resins is often preferred when the unsubstituted amide is desired because cleavage directly gives the C-terminal amide. When an N-methylamide is desired, such can be generated from an N-methyl BHA resin. Other single-substituted amides can be synthesized by the procedure set forth in W. Kornreich et al., Int. J. Peptide Protein Res., 25:414-420, 1985, and also in U.S. Pat. No. 4,701,499. Peptides having di-substituted amides at the C-terminus, such as N-piperidinyl, are preferably prepared via classical solution synthesis or by fragment condensation in solution.

Once synthesized, these tetrapeptides are readily purified using well known state of the art methods for short peptide purification, for example, reverse-phase high performance liquid chromatography (RP-HPLC), or other appropriate methods. Such purification is described in detail in J. Rivier et al., J. Chromatography, 288:303-328, 1984, and C. Miller and J. Rivier, Peptide Science, Biopolymers, 40:265-317 (1996), and specific examples of such purification following solid phase synthesis or the like are shown in U.S. Pat. No. 5,098,995.

A variety of assays may be employed to test whether the compounds of the invention exhibit high affinity and selectivity for the kappa opioid receptor. Receptor assays are well known in the art and kappa opioid receptors from several species have been cloned, as have mu and delta opioid receptors. Kappa opioid receptors as well as mu and delta opioid receptors are classical, seven-transmembrane spanning, G-protein coupled receptors. Although these cloned receptors readily allow a particular candidate compound, e.g., a peptide, to be screened, natural sources of mammalian opioid receptors are also useful for screening, as is well known in the art (Dooley C T et al. Selective ligands for the mu, delta, and kappa opioid receptors identified from a single mixture based tetrapeptide positional scanning combinatorial library. J. Biol. Chem. 273:18848-56, 1998). Thus, screening against both kappa and mu opioid receptors, whether of recombinant or natural origin, may be carried out in order to determine the selectivity of the compound(s) for the kappa relative to the mu opioid receptor.

Binding affinity refers to the strength of interaction between ligand and receptor. To demonstrate binding affinity for opioid receptors, the compounds of the invention can be evaluated using competition binding studies. These studies can be performed using cloned kappa and mu opioid receptors expressed in stable transfected cell lines or naturally occurring opioid receptors from a receptor-enriched tissue source, as noted above. In these studies, the test compounds (unlabeled ligands) are used at increasing concentrations to displace the specific binding of a radiolabeled ligand that has high affinity and selectivity for the receptor studied. Tritiated U-69,593 and DAMGO can be used as ligands in kappa and mu opioid receptor studies, respectively. Both ligands are commercially available (NEN-Dupont). DAMGO is an acronym for [D-Ala², MePhe⁴, Gly-ol⁵]-enkephalin. The affinity of the radioligands is defined by the concentration of radioligand that results in half-maximal specific binding (K_{D}) in saturation studies. The affinity of the test compound (unlabeled or cold ligand) is determined in competition binding studies by calculating the inhibitory constant (Kᵢ) according to the following formula:

Kᵢ=IC₅₀/[1+(F/K_{D})]

where IC₅₀=Concentration of the cold ligand that inhibits 50% of the specific binding of the radioligand

F=free radioligand concentration

K_{D}=affinity of the radioligand determined in saturation studies.

When performing these assays under specific conditions with relatively low concentrations of receptor, the calculated Kᵢ for the test compound is a good approximation of its dissociation constant K_{D}, which represents the concentration of ligand necessary to occupy one-half (50%) of the binding sites. A low Kᵢ value in the nanomolar and subnanomolar range is considered to identify a high affinity ligand at opioid receptors. Preferred analogs have a Kᵢ for kappa opioid receptor of about 10 nanomolar (nM) or less, whereas more preferred analogs have a Kᵢ of about 1 nM or less. High affinity compounds are preferred, in order to: (1) enable the use of relatively low doses of drug, which minimizes the likelihood of side effects due to low affinity interactions, and (2) potentially reduce the cost of manufacturing a dose since a correspondingly smaller amount of a higher affinity compound would be required to produce the desired therapeutic effect, assuming equal absorption, distribution, metabolism, and excretion. Preferred analogs also have a Kᵢ for the mu opioid receptor of about 1 micromolar (uM) or more, whereas more preferred analogs have a Kᵢ for the mu opioid receptor of about 10 uM or more. For the purpose of identifying useful compounds of the invention, the IC₅₀, under appropriate assay conditions, is a useful approximation of the Kᵢ.

These binding assays employing kappa opioid receptors and mu opioid receptors are straightforward to perform and can be readily carried out with large numbers of compounds to determine whether such compounds are kappa opioid receptor-selective and have high affinity. Such binding assays can be carried out in a variety of ways as well known to one of skill in the art, and one detailed example of an assay of this general type is set forth in Young E A et al. [³H]Dynorphin A binding and kappa selectivity of prodynorphin peptides in rat, guinea-pig and monkey brain. Eur. J. Pharmacol. 121:355-65, 1986.

A variety of assays may be employed to test whether the compounds of the invention exhibit low inhibitory activity at CYP450 enzymes in general and at CYP3A4 in particular. Enzyme assays are well known in the art and CYP450 enzymes from several species have been cloned. Although these cloned enzymes readily allow a particular candidate compound, e.g., a peptide, to be screened, natural sources of CYP450 enzymes, e.g., liver microsomes, are also useful for screening, as is well known in the art. Preferred analogs have a Kᵢ for CYP450 enzymes of about 10 micromolar (uM) or more, whereas more preferred analogs have a Kᵢ for the mu opioid receptor of about 100 uM or more. For the purpose of identifying useful compounds of the invention, the IC₅₀ is a useful approximation of the Kᵢ.

The present invention is further described by the example which follows. This example, however, is not to be construed as being limiting in any way to either the spirit or scope of the present invention which is described by the claims at the end hereof.

### Example 1

Peptide No. 1, having the formula: H-D-Phe-D-Phe-D-Nle-D-Arg-NH-4-picolyl-N-oxide, is appropriately synthesized as well known in the peptide synthesis art, particularly in view of the synthesis of peptides such as H-D-Phe-D-Phe-D-Nle-D-Arg-NH-4-picolyl as disclosed in U.S. Pat. No. 5,965,701. The structure of Peptide No. 1 is as follows:

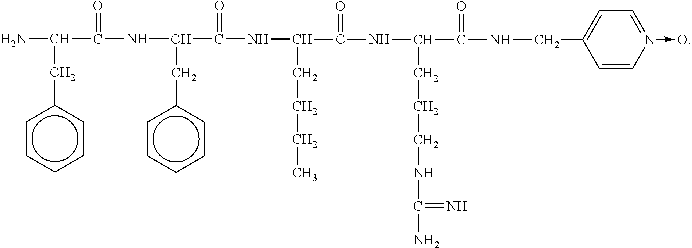

Binding assays with guinea pig and rat brain membranes containing KOR and MOR, respectively, are carried out as mentioned hereinbefore. The KOR binds Peptide No. 1 with high affinity as determined by the competitive displacement of bound radioligand, and the IC₅₀ is determined to be about 6.3 nM (Table 1). The difference in affinity is dramatic compared to MOR where the IC₅₀ is too high to determined under the conditions of the assay, since a maximal binding inhibition of only 15.5% was measured (Table 2). Thus, Peptide No. 1 binds more strongly to KOR than to MOR by a factor of much greater than 1,000. The bioactivity of Peptide 1 at the KOR was demonstrated in a functional second messenger assay, using Gᵢ-mediated inhibition of adenylate cyclase (Table 3), which is measured with cyclic AMP (cAMP) assays, as are well known in the art.

TABLE 1
Displacement of Kappa Opioid Receptor Binding by Peptide1
Kappa Opioid Receptor Binding
(guinea pig brain)

| | | IC₅₀ |
|---|---|---|
| | Pct Max | (nM) |
| Sample 1 | 117.3 | 9.4 |
| Sample 2 | 105.6 | 4.2 |
| Sample 3 | 105.2 | 2.6 |
| Mean | 109.4 | 6.3 |

TABLE 2
Lack of Displacement of Mu Opioid Receptor Binding by Peptide1
Mu Opioid Receptor Binding
(rat brain)

| | | IC₅₀ |
|---|---|---|
| | Pct Max | (nM) |
| Sample 1 | 15.5 | No Fit |

TABLE 3
Bioactivity of Peptide 1 at Kappa Opioid Receptors
Kappa Opioid Receptor Activity
(cAMP, mouse brain)

| | | EC₅₀ |
|---|---|---|
| | Pct Max | (nM) |
| Sample 1 | 92.46 | 0.30 |
| Sample 2 | 110.41 | 0.57 |
| Mean | 101.43 | 0.42 |

One of the selected peptides has been further specifically subjected to in vivo testing for determination of analgesic activity. The in vivo testing is carried out using a mouse writhing test (WT) that is well-suited for determining the length of duration of antinociceptive biological activity. This test is described in detail in an article by G. A. Bentley et al., Br. J. Phamac., 73:325-332, 1981, and it employs conscious male ICR mice which are purchased from Harlan and which weigh between 20 and 30 grams. The mice are fasted for 12 to 16 hours prior to beginning the test. The nociceptive behavior, i.e. writhing, to be monitored is induced by the intraperitoneal (i.p.) administration of dilute acetic acid. 10 milliliters of 0.6% aqueous acetic acid is used per kg of body weight. Writhing is scored during the 15 minutes following acetic acid administration. In general, compounds are tested at 3 to 4 increasing doses, given by intravenous route, and at one unique pretreatment time (−5 minutes before acetic acid injection). This step is used to determine the potency (WT-ED₅₀) as well as a submaximal effective dose (about 80-90% antinociception. Throughout the test, a control group of mice are used which are administered only the vehicle without the candidate peptide. The number of writhes are counted over a 15-minute period, starting from the time of acetic acid injection, and bioactivity, i.e. antinociception, is expressed as a percentage, as described in U.S. Pat. No. 5,965,701.

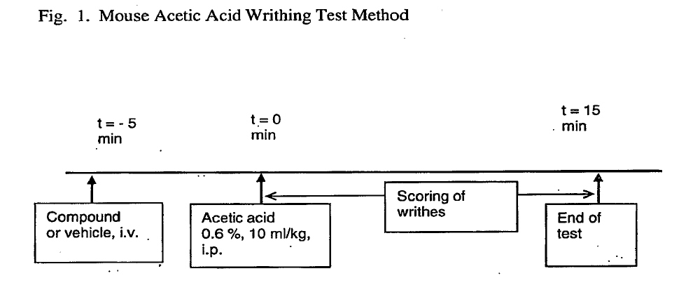

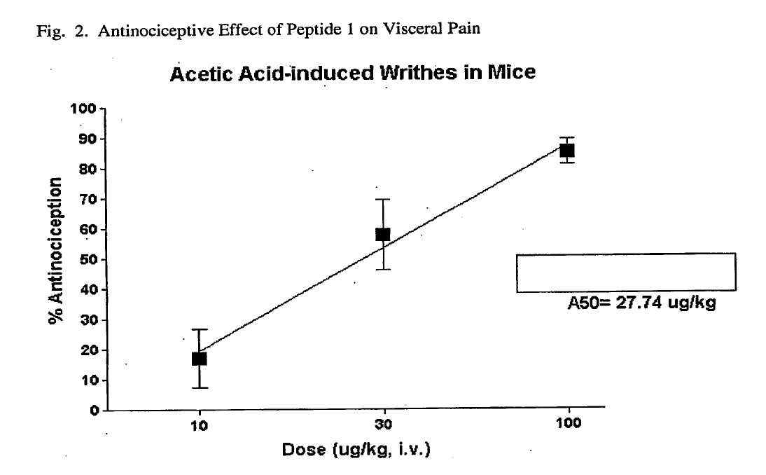

Testing of the peptide in the mouse acetic acid writhing assay (as described hereinafter, and illustrated in FIG. 1) shows an A₅₀ of about 0.027 mg/kg after intravenous administration (FIG. 2; 95% confidence limits: 0.019 to 0.040; R²=0.603). Testing of Compound 1 on several cytochrome P450 enzyme activities demonstrated an absence of significant inhibitory activity (Table 4), which is particularly notable for P450 3A4 isozymes, which were observed to be inhibited by related compounds lacking only the N-oxide substitution on the C terminal amide.

TABLE 4
Effect of Compound 1 on Cytochrome P450 Enzyme Activities

| | |
|---|---|
| Pct Max | IC₅₀ |

P450 3A4/BQ Inhibition

| | | |
|---|---|---|
| Sample 1 | 15 | No Fit |
| Sample 2 | 21 | No Fit |
| Mean | 18 | |

P450 3A4/BFC Inhibition

| | | |
|---|---|---|
| Sample 1 | 22 | No Fit |
| Sample 2 | 17 | No Fit |
| Sample 3 | 40 | |
| Mean | 26 | |

P450 2C9 Inhibition

| | | |
|---|---|---|
| Sample 1 | 21 | >10,000 |
| Sample 2 | 31 | >10,000 |
| Mean | 26 | |

P450 1A2 Inhibition

| | | |
|---|---|---|
| Sample 1 | 15 | No Fit |
| Sample 2 | 10 | No Fit |
| Mean | 13 | |

The opioid peptides are useful as analgesics and for other pharmacological applications to treat pathologies associated with the KOR system. They exhibit advantages over mu agonist painkillers, e.g. morphine which has undesirable effects, such as constipation, respiratory depression and itching. Peripheral effects are measured using the mouse writhing test (WT) described previously.

Because these peptides bind strongly to the KOR, they are also useful in in vitro assays for studying receptors and for determining what receptors may be present in a particular tissue sample. Thus, they are useful for diagnosis in this respect and potentially also for in vivo diagnosis.

Generally, these opioid peptides can be used to achieve antinociception in treating visceral pain and also to treat rheumatoid arthritis. They are particularly useful in treating abdominal postsurgery symptoms such as digestive disorders and pain. They are also considered to be effective to treat IBS, bladder instability, incontinence, and other indications where local inflammation results in pain states in the gut or in other viscera, e.g. inflammatory bowel disease (IBD) and dysmenorrhea. The opioid peptide's ability to lower immune response might be advantageous in combating IBD and other indications, such as autoimmune diseases. Administration of the peptides can be employed to produce local analgesic activity in respect of both acute and chronic inflammatory conditions. They can be used to treat digestive ileus having symptoms such as bloating, nausea or intestinal transit inhibitions associated with pain, e.g. bowel obstruction possibly caused by spastic contractions. The opioid peptides are also effective in producing peripheral analgesia, and they can be targeted to relieve post-operative pain, as well as chronic pain, such as that caused by inflammation of gastrointestinal and visceral tissues, and also to give relief during withdrawal from drug addiction. These compounds can also be used to treat pruritis (itch) associated with a variety of conditions, such as uremic pruritis in patients undergoing hemodialysis, and can further be used to induce aquaresis in conditions of water and sodium imbalance, e.g., euvolemic hyponatremia, which occurs when total body water increases with little increase in sodium, most often associated with conditions such as the syndrome of inappropriate antidiuretic hormone (SIADH), adrenal insufficiency, pulmonary disorders, hypothyroidism, certain cancers and the use of certain drugs (such as some antidepressants). The compounds can also be used to produce aquaresis to treat the imbalance of salt and water in disorders such as congestive heart failure and liver cirrhosis.

The compounds of the invention may be administered in the form of pharmaceutically acceptable, nontoxic salts, such as acid addition salts, as well known in this art. Illustrative of such acid addition salts are hydrochloride, hydrobromide, sulphate, phosphate, nitrate, oxalate, fumarate, gluconate, tannate, pamoate, maleate, acetate, citrate, benzoate, succinate, alginate, malate, ascorbate, tartrate and the like. If the active ingredient is to be administered in tablet form, the tablet may contain a pharmaceutically-acceptable, nontoxic diluent which includes a binder, such as tragacanth, corn starch or gelatin. Intravenous administration in isotonic saline, phosphate buffer, mannitol or glucose solutions may also be effected.

The pharmaceutical compositions will usually contain an effective amount of the peptide in conjunction with a conventional, pharmaceutically-acceptable carrier or diluent. Generally, the composition will contain an antinociceptive amount, i.e. an amount which is effective to block pain. Usually, the dosage will be from about 1 microgram to about 10 milligrams of the peptide per kilogram of the body weight of the host when given intravenously. The compositions may be administered as needed; for example, they may be administered repeatedly at 3-6 hour intervals. The nature of these compounds may possibly permit effective oral administration; however, oral dosages might be higher. If desirable to deliver the opioid peptide over prolonged periods of time, for example, for periods of one week or more from a single administration, slow release, depot or implant dosage forms may be utilized. For example, a suitable, slow-release depot formulation for injection may contain the peptide or a salt thereof dispersed or encapsulated in a slow-degrading, non-toxic or non-antigenic polymer, such as a polylactic acid/polyglycolic acid polymer, as described in U.S. Pat. No. 3,773,919. It is also known that administration by slow-release can be accomplished via a silastic implant, or using buccal patches, as have been described in the art.

These compounds can be administered to mammals intravenously, subcutaneously, intramuscularly, percutaneously, intranasally, intrapulmonarily, intrarectally or intravaginally, to achieve antinociception, such as to reverse gastrointestinal transit inhibition induced by peritoneal irritation. They may be so used for alleviation of post-operative pain. Effective dosages will vary with the form of administration and the particular species of mammal being treated. An example of one typical dosage form is a bacteriostatic water solution at a pH of about 3 to 8, e.g. about 6, containing the peptide, which solution is continuously administered parenterally to provide a dose in the range of about 0.3 micrograms to 3 mg/kg of body weight per day. These compounds are considered to be well-tolerated in vivo, and they are considered to be particularly well-suited for administration by subcutaneous injection in a bacteriostatic water solution or the like.

For nasal administration, the peripherally selective kappa opioid agonists can be formulated as aerosols. The term “aerosol” includes any gas-borne suspended phase of the compounds of the instant invention which is capable of being inhaled into the bronchioles or nasal passages. Specifically, aerosol includes a gas-borne suspension of droplets of the compounds of the instant invention, as may be produced in a metered dose inhaler or nebulizer, or in a mist sprayer. Aerosol also includes a dry powder composition of a compound of the instant invention suspended in air or other carrier gas, which may be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al. (1992) J. Pharmacol. Toxicol. Methods 27:143-159.

Parenteral administration of the formulations of the present invention includes intravenous, subcutaneous, intramuscular and transdermal administrations.

Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous, and thereby formulated for delivery by injection, infusion, or using implantable pumps. For intravenous, subcutaneous, and intramuscular administration, useful formulations of the invention include microcapsule preparations with controlled release properties (R. Pwar et al. Protein and peptide parenteral controlled delivery. Expert Opin Biol Ther. 2004 4(8):1203-12) or encapsulation in liposomes, with a preferred form being polyethylene-coated liposomes, which are known in the art to have an extended circulation time in the vasculature (e.g., Koppal, T. “Drug delivery technologies are right on target”, Drug Discov. Dev. 6, 49-50, 2003).

Preparations for transdermal delivery are incorporated into a device suitable for said delivery, said device utilizing, e.g., iontophoresis (Kalia Y N et al. Iontophoretic drug delivery. Adv Drug Deliv Rev. 56:619-58, 2004) or a dermis-penetrating surface (Prausnitz M R. Microneedles for transdermal drug delivery. Adv Drug Deliv Rev. 56:581-7, 2004), such as are known in the art to be useful for improving the transdermal delivery of drugs. An electrotransport device and methods of operating same are disclosed in U.S. Pat. No. 6,718,201. Methods for the use of iontophoresis to promote transdermal delivery of peptides are disclosed in U.S. Pat. No. 6,313,092 and U.S. Pat. No. 6,743,432. Herein the terms “electrotransport”, “iontophoresis”, and “iontophoretic” are used to refer to the delivery through a body surface (e.g., skin) of one or more pharmaceutically active compounds of the instant invention by means of an applied electromotive force to an agent-containing reservoir. The agent may be delivered by electromigration, electroporation, electroosmosis or any combination thereof. Electroosmosis has also been referred to as electrohydrokinesis, electro-convection, and electrically induced osmosis. In general, electroosmosis of a compound into a tissue results from the migration of solvent in which the compound is contained, as a result of the application of electromotive force to the therapeutic species reservoir, i.e., solvent flow induced by electromigration of other ionic species. During the electrotransport process, certain modifications or alterations of the skin may occur such as the formation of transiently existing pores in the skin, also referred to as “electroporation”. Any electrically assisted transport of species enhanced by modifications or alterations to the body surface (e.g., formation of pores in the skin) are also included in the term “electrotransport” as used herein. Thus, as used herein, applied to the compounds of the instant invention, the terms “electrotransport”, “iontophoresis” and “iontophoretic” refer to (1) the delivery of charged agents by electromigration, (2) the delivery of uncharged agents by the process of electroosmosis, (3) the delivery of charged or uncharged agents by electroporation, (4) the delivery of charged agents by the combined processes of electromigration and electroosmosis, and/or (5) the delivery of a mixture of charged and uncharged agents by the combined processes of electromigration and electroosmosis. Electrotransport devices generally employ two electrodes, both of which are positioned in close electrical contact with some portion of the skin of the body. One electrode, called the active or donor electrode, is the electrode from which the therapeutic agent is delivered into the body. The other electrode, called the counter or return electrode, serves to close the electrical circuit through the body. In conjunction with the patient's skin, the circuit is completed by connection of the electrodes to a source of electrical energy, e.g., a battery, and usually to circuitry capable of controlling current passing through the device.

Depending upon the electrical charge of the compound to be delivered transdermally, either the anode or cathode may be the active or donor electrode. Thus, if the compound to be transported is positively charged, e.g., the compound exemplified in Example 1 herein, then the positive electrode (the anode) will be the active electrode and the negative electrode (the cathode) will serve as the counter electrode, completing the circuit. However, if the compound to be delivered is negatively charged, then the cathodic electrode will be the active electrode and the anodic electrode will be the counter electrode. Electrotransport devices additionally require a reservoir or source of the therapeutic agent that is to be delivered into the body. Such drug reservoirs are connected to the anode or the cathode of the electrotransport device to provide a fixed or renewable source of one or more desired species or agents. Each electrode assembly is comprised of an electrically conductive electrode in ion-transmitting relation with an ionically conductive liquid reservoir which in use is placed in contact with the patient's skin. Gel reservoirs such as those described in Webster (U.S. Pat. No. 4,383,529) are the preferred form of reservoir since hydrated gels are easier to handle and manufacture than liquid-filled containers. Water is by far the preferred liquid solvent used in such reservoirs, in part because the salts of the preferred peptide compounds of the instant invention are water soluble and in part because water is non-irritating to the skin, thereby enabling prolonged contact between the hydrogel reservoir and the skin. Examples of reservoirs and sources include a pouch as described in U.S. Pat. No. 4,250,878, a pre-formed gel body as disclosed in U.S. Pat. No. 4,382,529, and a glass or plastic container holding a liquid solution of the drug, as disclosed in the figures of U.S. Pat. No. 4,722,726. For electrotransport, the peptides of the instant invention can be formulated with flux enhancers such as ionic surfactants (e.g., U.S. Pat. No. 4,722,726) or cosolvents other than water (e.g., European Patent Application 278,473). Alternatively the outer layer (i.e., the stratum corneum) of the skin can be mechanically disrupted prior to electrotransport delivery therethrough (e.g., U.S. Pat. No. 5,250,023).

Peripherally selective kappa opioid agonists that are well suited for electrotransport can be selected by measuring their electrotransport flux through the body surface (e.g., the skin or mucosa), e.g., as compared to a standardized test peptide with known electrotransport flux characteristics, e.g. thyrotropin releasing hormone (R. Burnette et al. J. Pharm. Sci. (1986) 75:738) or vasopressin (Nair et al. Pharmacol Res. 48:175-82, 2003). Transdermal electrotransport flux can be determined using a number of in vivo or in vitro methods well known in the art. In vitro methods include clamping a piece of skin of an appropriate mammal (e.g., human cadaver skin) between the donor and receptor compartments of an electrotransport flux cell, with the stratum corneum side of the skin piece facing the donor compartment. A liquid solution or gel containing the drug to be delivered is placed in contact with the stratum corneum, and electric current is applied to electrodes, one electrode in each compartment. The transdermal flux is calculated by sampling the amount of drug in the receptor compartment. Two successful models used to optimize transdermal electrotransport drug delivery are the isolated pig skin flap model (Heit M C et al. Transdermal iontophoretic peptide delivery: in vitro and in vivo studies with luteinizing hormone releasing hormone. J. Pharm. Sci. 82:240-3, 1993), and the use of isolated hairless skin from hairless rodents or guinea pigs, for example. See Hadzija B W et al. Effect of freezing on iontophoretic transport through hairless rat skin. J. Pharm. Pharmacol., 44, 387-390, 1992. Preferred compounds of the invention for transdermal iontophoretic delivery will have one, or most preferably, two charged nitrogens, to facilitate their delivery.

Other useful transdermal delivery devices employ high velocity delivery under pressure to achieve skin penetration without the use of a needle. Transdermal delivery can be improved, as is known in the art, by the use of chemical enhancers, sometimes referred to in the art as “permeation enhancers”, i.e., compounds that are administered along with the drug (or in some cases used to pretreat the skin, prior to drug administration) in order to increase the permeability of the stratum corneum, and thereby provide for enhanced penetration of the drug through the skin. Preferred chemical penetration enhancers are compounds that are innocuous and serve merely to facilitate diffusion of the drug through the stratum corneum, whether by passive diffusion or an energy-driven process such as electrotransport. See, for example, Meidan V M et al. Enhanced iontophoretic delivery of buspirone hydrochloride across human skin using chemical enhancers. Int. J. Pharm. 264:73-83, 2003.

Although the invention has been described with regard to its preferred embodiments, it should be understood that changes and modifications as would be obvious to one having the ordinary skill in this art may be made without departing from the scope of the invention which is set forth in the claims which are appended hereto. For example, other substitutions known in the art which do not significantly detract from the effectiveness of the peptides may be employed in the peptides of the invention. Other substituted D-Phe residues, such as (4Br)D-Phe or (2,4Cl₂)D-Phe, can be used in the 2-position. Both D-Lys(Bu) and D-Lys(Et₂) are considered to be equivalents of D-Ily and D-Arg(Et₂). The N-terminus of the tetrapeptide may be permethylated, as known in this art, if desired.

The disclosures of all U.S. patents hereinbefore mentioned are incorporated herein by reference. Particular features of the invention are emphasized in the claims which follow.

What is claimed is:

1. A compound comprising a metabolite of a synthetic peptide amide wherein (i) the metabolite can be formed from administration of the peptide amide to a mammal, (ii) the compound has an affinity for the kappa opioid receptor which is at least 1,000 times its affinity for the mu opioid receptor, (iii) the compound exhibits little or no cytochrome P450 enzyme inhibitory activity, and (iv) the peptide amide has the formula:
H-Xaa₁-Xaa₂-Xaa₃-Xaa₄-Q
wherein Xaa₁ is selected from the group consisting of D-Phe wherein the phenyl group is optionally substituted with NO₂, F, Cl or CH₃, D-Phe wherein the amino acid alpha carbon is methyl substituted, D-Tyr, D-Tic, D-Acp, D-2-Thi, or D-3-Thi; Xaa₂ is selected from the group consisting of D-Phe wherein the phenyl group is optionally substituted with NO₂, F, Cl, 3,4-dichloro or CH₃, D-1Nal, D-2Nal, D-Tyr or D-Trp; Xaa₃ is selected from the group consisting of D-Nle, D-Leu, D-Leu wherein the amino acid alpha carbon is methyl substituted, D-Hle, D-Met, D-Val, D-Phe or D-Acp; Xaa₄ is selected from the group consisting of D-Arg, D-Har, D-nArg, D-Lys, D-Lys(Ipr), D-Arg(Et₂), D-Har(Et₂), D-Amf(G), D-Dbu, D-Orn, D-Orn wherein the amino acid alpha carbon is methyl substituted, or D-Orn(Ipr), with G being H or amidino; and Q is NR₁R₂, piperidinyl, 4-hydroxy piperidinyl, 4-oxo piperidinyl, piperazinyl, 4-mono- or 4,4-di-substituted piperazinyl or delta-ornithinyl, with R₁ being substituted benzyl, 2-thiazolyl, 2-picolyl, 3-picolyl or 4-picolyl, R₂ being H or lower alkyl.

2. The compound of claim 1 wherein the metabolite is an N-oxide of the synthetic peptide amide.

3. The compound of claim 2 wherein Q is NHR₁, R₁ is 2-picolyl, 3-picolyl, or 4-picolyl.

4. The compound of claim 3 wherein the N-oxide is formed at the ring nitrogen of the picolyl moiety.

5. The compound of claims 1-4 wherein Xaa₁ and Xaa₂ are D-Phe, Xaa₃ is D-Nle or D-Leu, Xaa₄ is D-Arg or D-Orn.

6. The compound of claim 1 wherein the synthetic peptide amide is H-D-Phe-D-Phe-DNle-D-Arg-NH-4-picolyl optionally including or excluding any pharmaceutically acceptable counterions.

7. The compound of claim 6 wherein the compound is H-D-Phe-D-Phe-DNle-D-Arg-NH-4-picolyl N-oxide optionally including or excluding any pharmaceutically acceptable counterions.

8. A compound which is an N-oxide of a synthetic peptide amide having the formula:
H-Xaa₁-Xaa₂-Xaa₃-Xaa₄-Q-N-oxide
wherein Xaa₁ is selected from the group consisting of D-Phe wherein the phenyl group is optionally substituted with NO₂, F, Cl or CH₃, D-Phe wherein the amino acid alpha carbon is methyl substituted, D-Tyr, D-Tic, D-Acp, D-2-Thi, or D-3-Thi; Xaa₂ is selected from the group consisting of D-Phe wherein the phenyl group is optionally substituted with NO₂, F, Cl, 3,4-dichloro or CH₃, D-1Nal, D-2Nal, D-Tyr or D-Trp; Xaa₃ is selected from the group consisting of D-Nle, D-Leu, D-Leu wherein the amino acid alpha carbon is methyl substituted, D-Hle, D-Met, D-Val, D-Phe or D-Acp; Xaa₄ is selected from the group consisting of D-Arg, D-Har, D-nArg, D-Lys, D-Lys(Ipr), D-Arg(Et₂), D-Har(Et₂), D-Amf(G), D-Dbu, D-Orn, D-Orn wherein the amino acid alpha carbon is methyl substituted, or D-Orn(Ipr), with G being H or amidino; and Q is NR₁R₂, piperidinyl, 4-hydroxy piperidinyl, 4-oxo piperidinyl, piperazinyl, 4-mono- or 4,4-di-substituted piperazinyl or delta-ornithinyl, with R₁ being substituted benzyl, 2-thiazolyl, 2-picolyl, 3-picolyl or 4-picolyl, R₂ being H or lower alkyl; or any polymorphs of said compound.

9. The compound of claim 8 wherein Q is NHR₁, R₁ is 2-picolyl, 3-picolyl, or 4-picolyl, and the N-oxide is formed at the ring nitrogen of the picolyl moiety.

10. The compound of any of claims 8-9 wherein Xaa₁ and Xaa₂ are D-Phe, Xaa₃ is D-Nle or D-Leu, Xaa₄ is D-Arg or D-Orn.

11. The compound of claim 10 wherein the compound is H-D-Phe-D-Phe-DNle-D-Arg-NH-4-picolyl N-oxide optionally including or excluding any pharmaceutically acceptable counterions.

12. A pharmaceutical composition which comprises an antinociceptive amount of the compound according to any of claims 1-11 and a pharmaceutically acceptable liquid or solid carrier therefor.

13. A method of treatment which comprises administering a pharmaceutically effective amount of the compound of any of claims 1-11 wherein the method is effective (a) to achieve antinociception where there is visceral pain, rheumatoid arthritis, abdominal postsurgery symptoms or acute or chronic pain, or (b) to treat bladder instability, incontinence or digestive ileus, (c) to treat IBD or autoimmune diseases, or (d) to relieve pruritis, or (e) produce aquaresis in a condition where an imbalance of body sodium and water contributes to the symptoms of said condition.
